(19) United States
(12) Patent Application Publication
(10) Pub. No.: US 2025/0319078 A1
(43) Pub. Date: Oct. 16, 2025

(54) METHOD FOR SYNERGISTIC ENHANCEMENT OF REMYELINATION VIA MODULATION OF RXR AND A HETERODIMERIC PARTNER

(71) Applicants: Luke L. LAIRSON, San Diego, CA (US); Brittney A. BEYER, San Diego, CA (US); THE SCRIPPS RESEARCH INSTITUTE, La Jolla, CA (US)

(72) Inventors: Luke L. LAIRSON, San Diego, CA (US); Brittney A. BEYER, San Diego, CA (US)

(21) Appl. No.: 18/866,591

(22) PCT Filed: May 18, 2023
(86) PCT No.: PCT/US2023/022725

§ 371 Date: Nov. 18, 2024

Related U.S. Application Data
(60) Provisional application No. 63/364,937, filed on May 18, 2022.

(51) Int. Cl. A61K 31/4545; A61K 31/138; A61K 31/18; A61K 31/192; A61K 31/341; A61K 31/416; A61K 31/496; A61K 31/5375; A61P 25/00

(52) U.S. Cl. CPC A61K 31/4545; A61K 31/138; A61K 31/18; A61K 31/192; A61K 31/341; A61K 31/416; A61K 31/496; A61K 31/5375; A61P 25/00

(57) ABSTRACT

Disclosed herein are methods and compositions for treating a demyelinating disease, such as multiple sclerosis, by administering to a subject suffering from the disease at least one Retinoid X receptor gamma (RXRy) agonist and at least one member selected from a Liver X Receptor (LXR) antagonist, CYP51 inhibitor, TM7SF2 inhibitor, EBP inhibitor, and combinations thereof.

The present application claims the benefit of priority to U.S. Provisional Patent Application No. 63/364,937 filed on May 18, 2022, which application is incorporated in its entirety as if fully set forth herein.

Remyelination is a process facilitated by the differentiation of oligodendrocyte progenitor cells (OPCs) into functional, myelin sheath-forming oligodendrocytes (OLs). In multiple sclerosis (MS), inflammatory lesions progressively fail to remyelinate, contributing to irreversible axonal loss and neurodegeneration that correlates with clinical disability. MS is a debilitating autoimmune disease that is characterized by episodes of focal inflammation leading to the primary demyelination of axons, neuronal dysfunction and ultimately axonal loss¹²⁻¹⁴. Inflammatory and immune attacks in MS target oligodendrocytes (OLs), which are the cell type within the central nervous system (CNS) that produce and maintain the myelin sheaths surrounding axons. Approved treatments for MS primarily consist of anti-inflammatory and immunomodulatory drugs. Despite the beneficial impact that these drugs have on disease severity and frequency of relapse, immune targeting alone ultimately fails and MS invariably progresses to a state of chronic demyelination, permanent disability, and reduced lifespan¹⁸.

As the most prevalent demyelinating disease of the CNS, MS affects approximately 2.5 million people worldwide¹². It is the most common neurological disease of young adults in North America, with typical onset around the third decade of life^{15, 16}. Hence, the socioeconomic burden associated with MS is significant due to the high cost of treatment and additional care associated with neurological disability¹⁷.

The development of regenerative therapeutics to address the observed impairment of remyelination that occurs during progressive phases of MS can be recognized as a major unmet medical need. Remyelination is a regenerative process that persists throughout adulthood in mammals and involves the differentiation of oligodendrocyte progenitor cells (OPCs) into myelinating oligodendrocytes (OLs). The ability of OPCs to facilitate remyelination diminishes with disease progression, age, and in the presence of inhibitory milieu.

Genetic fate mapping studies have shown that OL progenitor cells (OPCs), a highly abundant population of cells that persist throughout adulthood within the mammalian CNS, give rise to remyelinating OLs^{19, 20}. OPCs, which are identified by distinct lineage markers including PDGFRα (platelet-derived growth factor receptor alpha) and NG2 (neural/glial antigen 2), are estimated to make up ˜5% of total cells within the CNS²¹. In response to demyelination, OPCs become activated to drive remyelination, a regenerative process that is defined by the recruitment and subsequent differentiation of OPCs, resulting in the generation of mature, myelinating OLs.²²⁻²⁷. Based on the abundant numbers of OPCs that are often observed to be present in MS lesions¹, it is widely believed that inhibition of OPC differentiation plays a major role in disease progression²⁸⁻³². OLs that survive demyelination can participate in myelin repair, but they do so in a limited capacity compared to newly-generated OLs³³⁻³⁵.

### SUMMARY

With an increasing rate of MS incidence throughout the world, along with a rise in disability within aging populations, there is a need for regenerative therapies to promote repair and functional recovery^{2, 3}.

Accordingly, the present disclosure provides, in embodiments, a method for the treatment of a demyelinating disease in a subject suffering therefrom. The method comprises administering to the subject at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof.

In additional embodiments, the present disclosure provides a method for remyelination of demyelinated axons in a subject. The method comprises administering to the subject at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof.

### BRIEF DESCRIPTION OF THE DRAWINGS

FIG. 1. Chemical structures of tamoxifen, tasin-1, and bexarotene.

FIGS. 2A-2G. Synergistic differentiation-inducing effect RXR agonism on select compounds that drive accumulation of 8,9-unsaturated sterols. a, Double gradient heat map showing Bexarotene+/−Tasin-1 activity as average (n=3) % of T3-induced MBP+OLs (blue=0%; yellow=DMSO baseline; red=largest value). b, Double gradient heat map showing Bexarotene+/−Tamoxifen activity as average (n=3) % of T3-induced MBP+OLs (blue=0%; yellow=DMSO baseline; red=largest value). c, Comparative impact on % T3-induced MBP+OLs by tasin-1 at range of doses vs. tasin-1+bexarotene (500 nM). d, Comparative impact on % T3-induced MBP+OLs by tasin-1 (50 or 100 nM) vs. with bexarotene (500 nM) added. e, Comparative impact on % T3-induced MBP+OLs by tamoxifen at range of doses vs. tasin-1+bexarotene (500 nM). f, Comparative impact on % T3-induced MBP+OLs by tamoxifen (200 or 400 nM) vs. with bexarotene (500 nM) added. g, Representative images showing impact on MBP+OLs following 6 d treatment with T3 (1 μM), bexarotene (500 nM), tasin-1 (150 nM), tamoxifen (300 nM) or bexarotene+tasin-1 or tamoxifen. “***”, and “****” represent p<0.001 and p<0.0001; respectively.

FIGS. 3A and 3B. Co-treatment of bexarotene with inhibitors of CYP51 or TM7SF2 as functional inducers of 8, 9 unsaturated sterols. a, Double gradient heat maps showing impact of bexarotene+/−ketoconazole or amorolifine as average (n=3) of % T3-induced MBP+OL, (blue=0%; yellow=30%; red=80%). b, Comparative impact on % T3-induced MBP+OLs by ketoconazole (3.3 μM), amorolifine (130 nM), +/−bexarotene (0.8 μM).

FIGS. 4A, 4B, 4C, and 4D. Impact on illustrative bexarotene combinations via modulation of LXR. (A): Comparative impact on % T3-induced MBP+OLs by Bexarotene, Tasin-1 or a combination of both +/−300 nM LXR623. (B): Comparative impact on % T3-induced MBP+OLs by Bexarotene, Tasin-1 or a combination of both +/−300 nM GSK2033. “****” represents p<0.0001. (C and D) Concentration gradient data from graphs in (A) and (B), respectively, are derived.

FIGS. 5A and 5B. (A) Impact on localization of LXR induced by Tasin-1 or Tamoxifen treatment. (B) Line profile showing fluorescent intensities of individual cells (n=10) across an 18.5 μm line bisecting each cell following 6 days of treatment with tasin-1 (150 nM).

FIG. 6. Western blot-based analysis of OPC differentiation using induced MBP protein expression as readout.

FIG. 7. An element of the cholesterol biosynthesis pathway.

FIG. 8. A derivative of the cholesterol pathway (Zymosterol, an 8,9-unsaturated sterol) phenocopies tasin-1, tamoxifen and LXR agonists.

FIGS. 9A and 9B. Impact of bexarotene supplementation on 8, 9 saturated vs. unsaturated sterols. Follicular fluid meiosis-activating sterol (FF-MAS), but not desmosterol (saturated at 8, 9 position), combines with bexarotene (RXRg agonist) to phenocopy tasin-1, tamoxifen, or an LXR antagonist. (A) Double gradient heat maps showing impact of LXR antagonist GSK 2033 or LXR agonist LXR623 on bexarotene+/−FF-MAS-induced activity, represented as average (n=3) of % T3-induced MBP+OL, (blue=0%; yellow=10%; red=30%). (B) Double gradient heat maps showing impact of LXR antagonist GSK 2033 or LXR agonist LXR623 on bexarotene+/−Desmosterol-induced activity, represented as average (n=3) of % T3-induced MBP+OL, (blue=0%; yellow=10%; red=30%).

FIG. 10. Gradient heat map showing that SR9243, a structurally orthogonal LXR antagonist, phenocopies GSK2033, FFMAS, tasin-1 and tamoxifen (blue=0%; yellow=20%; red=40%).

### DETAILED DESCRIPTION

The present disclosure is premised in part upon a combination of agents (i.e., differentiation-inducing and maturation-inducing) that surprisingly induces overall OPC differentiation and maturation. Agents that promote functional OL maturation translate to disease-modifying treatments for demyelinating diseases. Furthermore, co-administration of agents that modulate multiple, relevant remyelination targets result in superior treatments for such diseases.

Illustrating these advantages, per exemplary embodiments described herein, are synergistic, remyelinating-enhancing combinations of bexarotene, a selective retinoid X receptor (RXR) agonist, with inhibitors of select enzymes within the cholesterol biosynthetic pathway. The inhibition of CYP51, TM7SF2 or EBP results in the accumulation of 8,9-unsaturated sterol intermediates that, by previously unresolved mechanisms, signal to enhance remyelination (FIG. 7). There was observed a potent, synergistic enhancement of remyelination by activating RXR while utilizing agents that regulate the production of endogenous metabolites which modulate a likely downstream target, the liver X receptor (LXR). In one embodiment, for example, the most potent synergistic impact resulted from co-administration of an RXR agonist with select inhibitors of EBP, such as TASIN-1 or tamoxifen. Each of these compounds are minimally active in enhancing OPC differentiation alone, but upon activation of RXR, their ability to drive functional OL maturation, and thereby remyelination, is drastically enhanced.

### Definitions

As used herein, and unless otherwise clear from context or specified to the contrary, the term “compound” is inclusive in that it encompasses a compound or a pharmaceutically acceptable salt thereof.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

As used herein, the term “oligodendrocyte precursor cell” or “OPC” refers to an undifferentiated progenitor cell with the capacity to self-renew and differentiate into a myelinating oligodendrocyte. A “mature myelinating cell fate” refers to cell that is capable of forming myelin, e.g., a myelinating oligodendrocyte. “Differentiation” refers to the process by which a specialized cell type is formed from a less specialized cell type, for example, a myelinating oligodendrocyte from an OPC. In some embodiments, an OPC is identified by morphology and/or by the presence of a biomarker, e.g., PDGFR-α or NG2. In some embodiments, a myelinating oligodendrocyte is identified by morphology and/or by the presence of a marker, e.g., myelin basic protein (MBP), myelin oligodendrocyte glycoprotein (MOG), 2′3′-cyclic-nucleotide 3′ phosphodiesterase (CNP), galactocebroside (GalC), O1 antigen (O1), or O4 antigen (O4).

As used herein, the term “remyelination” refers to inducing an increased amount of myelin surrounding an axon, e.g., by administering an agent that induces the differentiation of oligodendrocyte precursor cells to a mature myelinating cell fate, as compared to the amount of myelin surrounding the axon in the absence of the agent being administered. In some embodiments, an agent stimulates “increased” myelination when the amount of myelin surrounding the axon in a sample (e.g., a brain tissue sample from a subject having a demyelinating disease) subsequent to administration of an agent that induces the differentiation of OPCs to a mature myelinating cell fate is at least about 5%, 10%, 15%, 20%, 30%, 40%, 50%, 60%, 70%, 80%, 90%, or more as compared to the amount of myelin surrounding the axon in the sample prior to administration of the agent. The amount of myelin surrounding an axon can be measured by any method known in the art, e.g., using magnetic resonance imaging (MRI). In some embodiments, an agent stimulates increased myelination when one or more characteristics of a demyelinating disease (e.g., multiple sclerosis) improves subsequent to administration of an agent that induces differentiation of OPCs to a mature myelinating cell fate as compared to the characteristic of the diseases prior to administration of the agent. As a non-limiting example, an agent is said to stimulate increased myelination in a subject having multiple sclerosis when the frequency and/or severity of inflammatory attacks decreases subsequent to administration of an agent as compared to the frequency and/or severity of inflammatory attacks prior to administration of the agent.

As used herein, the term “demyelinating disease” refers to a disease or condition of the nervous system characterized by damage to or loss of the myelin sheath of neurons. A demyelinating disease can be a disease affecting the central nervous system or a disease affecting the peripheral nervous system. Examples of demyelinating diseases include, but are not limited to, multiple sclerosis, idiopathic inflammatory demyelinating disease, transverse myelitis, Devic's disease, progressive multifocal leukoencephalopathy, optic neuritis, leukodystrophy, Guillain-Barre syndrome, chronic inflammatory demyelinating polyneuropathy, autoimmune peripheral neuropathy, Charcot-Marie-Tooth disease, acute disseminated encephalomyelitis, adrenoleukodystrophy, adrenomyeloneuropathy, Leber's hereditary optic neuropathy, or HTLV-associated myelopathy. In some embodiments, the demyelinating disease is multiple sclerosis.

The terms “treat”, “treating” and “treatment” refer to the amelioration or eradication of a disease or symptoms associated with a disease. In various embodiments, the terms refer to minimizing the spread or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Methods of Use

The present disclosure is premised in part upon the surprising and significant enhancement of OL maturation above levels achieved by monotherapy in a synergistic combination of at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof. The combination is useful, in some embodiments, in a method of treatment of a demyelinating disease in a subject suffering therefrom, comprising administering the combination to the subject.

In some embodiments, the RXRγ agonist is administered with an EBP inhibitor. Exemplary EBP inhibitors, per some embodiments, include tasin-1 and tamoxifen.

In other embodiments, the RXRγ agonist is administered with a CYP51 inhibitor. In an illustrative embodiment, the CYP51 inhibitor is ketoconazole.

In still additional embodiments, the RXRγ agonist is administered with a TM7SF2 inhibitor. An example of a TM7SF2 inhibitor, in one embodiment, is amorolfine.

Many inhibitors of EBP, CYP51, and TM7SF2 are suitable for use in the methods described herein and are known to the skilled person for inhibiting a narrow range of steps in the biosynthesis of cholesterol and inducing the accumulation of 8,9-unsaturated sterols. See D. Allimuthu et al., Cell Chem Biol. 26(4) (2019) 593-599.e4. and Z. Hubler et al., Nature 560 (2018) 372-376, which are incorporated herein by reference in their entireties.

In some embodiments, the RXRγ agonist is administered with an LXR antagonist. Examples of LXR antagonists, in various embodiments, include GSK2033, LXR623, and SR9243.

In an embodiment, optionally in combination with any other embodiment described herein, the RXRγ agonist is bexarotene.

In some embodiments, the demyelinating disease is a demyelinating disease of the central nervous system (CNS). Demyelinating diseases of the CNS include, in various embodiments, multiple sclerosis, neuromyelitis optica (Devic's disease), an idiopathic inflammatory demyelinating disease, a leukodystrophic disease, acute disseminated encephalomyelitis, optic neuritis, transverse myelitis, adrenoleukodystrophy, adrenomyeloneuropathy, central pontine myelinolysis, and a leukoencephalopathy. An illustrative demyelinating disease of the CNS is multiple sclerosis.

In other embodiments, the demyelinating disease is a demyelinating disease of the peripheral nervous system. Examples of such a disease includes Guillain-Barre syndrome, chronic inflammatory demyelinating polyneuropathy, anti-MAG peripheral neuropathy, Charcot-Marie-Tooth disease, Hereditary neuropathy with liability to pressure palsy, a copper deficiency-associated condition, and progressive inflammatory neuropathy. In various embodiment, the copper deficiency-associated condition is selected from peripheral neuropathy, myelopathy, and optic neuropathy.

Treatment of the disease comprises, in one embodiment, comprises retarding the rate of disease progression. Thus, therapeutic intervention as contemplated herein acknowledges that some demyelinating diseases may not be fully eradicated from the subject who is treated, and that acceptable therapy resides an extension of quality of life, motor skills, and the like, that would otherwise not occur but for the treatment as described herein. In other embodiments, treatment can result in the arresting of disease progression as determined, for example, by clinical assessment of symptoms and imaging techniques for direct observation of nervous system integrity. In still other embodiments, the methods described herein can comprise the reversing of disease progression, thereby restoring at least some functionality lost to the subject through disease. In additional embodiments, the treatment reduces the frequency and/or severity of disease symptoms, such as those suffered by a subject in multiple sclerosis relapse or flare-up, including new and old symptoms like fatigue, dizziness, balance and coordination difficulty, vision trouble, incontinence, numbing or tingling feelings, memory difficulty, and trouble with mental concentration.

In additional embodiments, optionally in combination with any other embodiment described herein, the present disclosure provides a method for remyelination of demyelinated axons in a subject. The method comprises administering to the subject at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof as described herein.

In some embodiments, the axons are partially demyelinated. In other embodiments, the axons are completely demyelinated. In still additional embodiments, the axons are in the central nervous system of the subject.

As illustrated by examples herein, the methods of the present disclosure provide an enhancement of OPC differentiation and/or OL maturation effected by an RXRγ agonist that acts synergistically in combination with an inhibitor of EBP, CYP51, or TM7SF2. The level of OL maturation is thereby enhanced more than the additive effects of the RXRγ agonist and inhibitor, respectively. The synergism, in various embodiments, can be defined by an enhancement of OL maturation that is about 2-fold, 3-fold-, 4-fold, 5-fold, 6-fold, 7-fold, 8-fold, 9-fold, 10-fold, 11-fold, 12-fold, 13-fold, 14-fold, or 15-fold greater than the sum of enhancements by the agents alone.

### Pharmaceutical Composition

The disclosure also provides, optionally for use in combination with the methods described herein, a pharmaceutical composition comprising a therapeutically effective amount of one or more compounds or a pharmaceutically acceptable salt described herein, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof as described herein, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

### Amounts

The “therapeutically effective amount” of a compound or a pharmaceutically acceptable salt thereof that is administered is governed by such considerations, and is the minimum amount necessary to induce oligodendrocyte progenitor cell (OPC) differentiation, to induce maturation of myelin sheath-forming oligodendrocytes (OLs), to remyelinate demyelinated axons, and combinations thereof. Such amount may be below the amount that is toxic to normal cells, or the subject as a whole. Generally, the initial therapeutically effective amount of each Retinoid X receptor gamma (RXRγ) agonist, Liver X Receptor (LXR) antagonist, CYP51 inhibitor, TM7SF2 inhibitor, EBP inhibitor, that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound of the present disclosure. In another embodiment, such dosage forms contain from about 50 mg to about 500 mg of a compound of the present disclosure. In yet another embodiment, such dosage forms contain from about 25 mg to about 200 mg of a compound of the present disclosure. In still another embodiment, such dosage forms contain from about 10 mg to about 100 mg of a compound of the present disclosure. In a further embodiment, such dosage forms contain from about 5 mg to about 50 mg of a compound of the present disclosure. In any of the foregoing embodiments the dosage form can be administered once a day or twice per day.

The combination of the agents RXRγ agonist and liver enzyme inhibitor as described herein surprisingly achieves a synergistic effect on remyelination, relative to the effect achieved by either agent alone. In some embodiments, an advantage resides in the ability to use suboptimal concentrations of either single agent, thereby reducing or eliminating toxicity or off-target concerns attributable to use of optimal concentrations of a single agent. In embodiments, the ratio of RXRγ agonist to liver enzyme inhibitor (i.e., CYP51, TM7SF2, or EBP inhibitor), is about 30:1 to about 1:5, about 25:1 to about 1:2, about 20:1 to about 1:1, about 15:1 to about 1.5:1, or about 10:1 to about 2:1. Exemplary ratios include about 20:1, 15:1, 10:1, 9:1, 8:1, 7:1, 6:1, 5:1, 4:1, 3:1, 2:1, 1:1, 1:2, 1:3, 1:4, and 1:5. The amounts of RXRγ agonist and liver enzyme inhibitor are chosen, in part, upon various factors known to the skilled clinician, including disease to be treated and health of the subject.

In embodiments wherein administration comprises administering a Liver X Receptor (LXR) antagonist, the ratio of RXRγ agonist to LXR antagonist is about 20:1 to about 1:0.5, about 15:1 to about 1:0.8, about 12:1 to about 1:1, about 10:1 to about 1:1.3, about 7:1 to about 1:1.5, about 5:1 to about 2:1. Exemplary ratios include about 20:1, 15:1, 14:1, 13:1, 12:1, 11:1, 10:1, 9:1, 8:1, 7:1, 6:1, 5:1, 4:1, 3:1, 2:1, and 1:1. An illustrative ratio of RXRγ agonist to LXR antagonist is about 3:1 or 2:1.

### Routes of Administration and Formulations

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In an embodiment, also encompassed are pharmaceutical compositions suitable for single unit dosages that comprise a compound of the disclosure or its pharmaceutically acceptable salt, and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxypropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical composition may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compound as described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

The following examples provide further illustrative embodiments of the present disclosure.

### EXAMPLES

### General Methods

Combination-based screening and evaluation of LXR modulators. Pairwise screening of bexarotene and partner compounds was done in matrices of 9-12 doses for each and transferred onto 3 separate 384-well plates. Immunofluorescence staining was quantified based on % MBP+OLs as described in B. A. Beyer et al., Nature Chemical Biology 14 (2018) 22-28. Heat maps were generated using Prism to highlight impact of the combinations relative to baseline controls.

Confocal imaging and analysis of LXR expression. Tasin-1, tamoxifen, bexarotene or bexarotene+tasin-1 or tamoxifen were transferred onto black, clear-bottom 384-well plates were coated with poly-d-lysine. OPCs were then seeded at a density of 1,000 cells per well, and the plates were incubated at 37° C. with 5% CO₂ for 6 d. On day 6, cells were then fixed for 20 min with 4% formaldehyde solution and stained with anti-LXRalpha/beta antibody (cat. #sc-271064, Santa Cruz Biotechnology) in 3% BSA, 0.3% Triton X-100 with overnight incubation at 4° C. The cells were washed and incubated with secondary antibody (Alexa Fluor 488 goat anti-mouse IgG, Life Technologies) and Hoechst 33342 (Invitrogen) for 1 h at room temperature. The cells were washed and plates were sealed and imaged using a Cellomics Cell Insight imaging reader (Thermo). Randomly-selected fields from 6 wells selected at random were imaged at 50× magnification using a Zeiss 780 confocal microscope. 10 cells within images from each treatment group were randomly selected for line profile analysis.

A line profile analysis of fluorescent intensities was performed by drawing an 18.5 μm line through the center of individual cells (n=10), from approximately 2-4 microns beyond the nuclear membrane on each side using the Fiji image processing platform. Fluorescence intensities over distance (μm) was graphed using Prism 9 software (GraphPad).

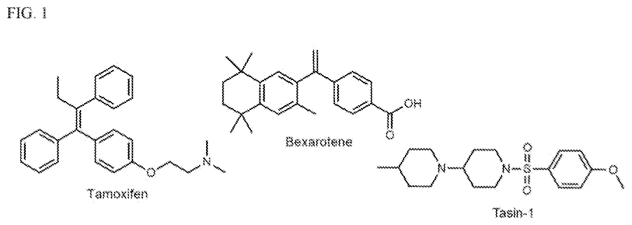

Using a cell-based phenotypic assay which quantifies the percentage of mature, myelin basic protein (MBP)-positive OLs, we identified compounds that enhance remyelination following RXR activation by bexarotene (FIG. 1). We evaluated various reported inhibitors of CYP51, TM7SF2 and EBP for their ability to enhance bexarotene-induced remyelination. We quantified activity based on immunofluorescence analysis of % MBP+OLs after 6 days of compound treatment, with vehicle/DMSO, clemastine and triiodothyronine (T3) controls on each plate using rat optic nerve derived OPCs.

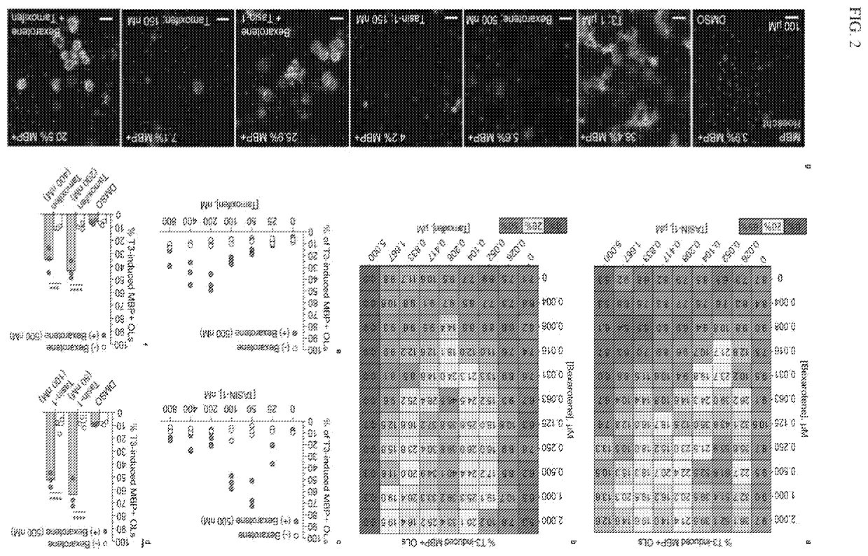

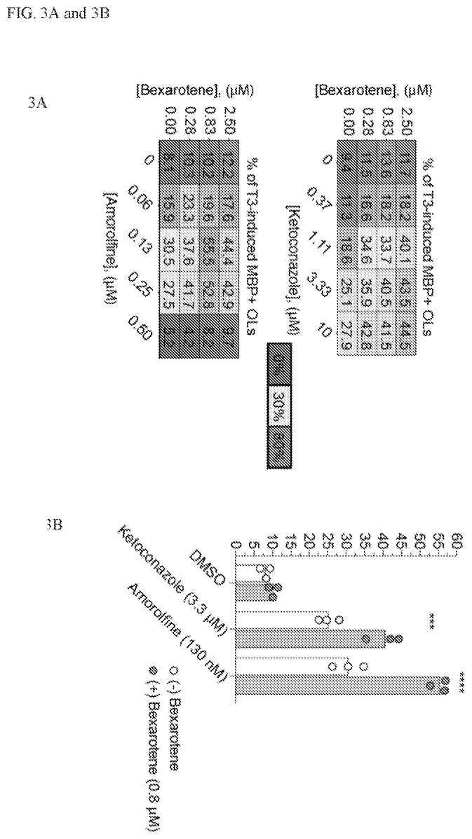

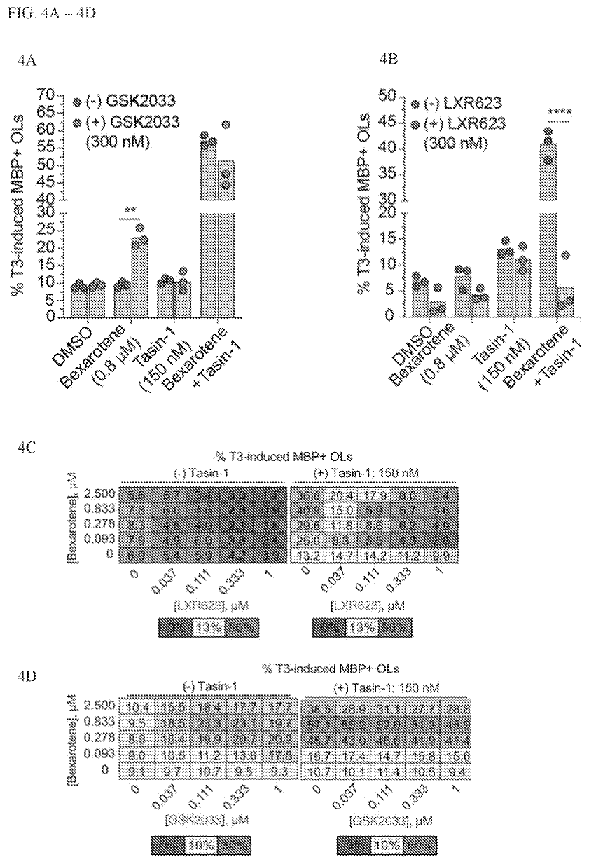

Inhibitors of the aforementioned enzymes demonstrated efficacy in driving remyelination following RXR activation (FIGS. 2, 3). Co-treatment with bexarotene and inhibitors of the most downstream of the three enzymes, EBP, resulted in the most robust impact on OL maturation, in some cases to levels that approached or exceeded T3-induced activity (FIG. 2).

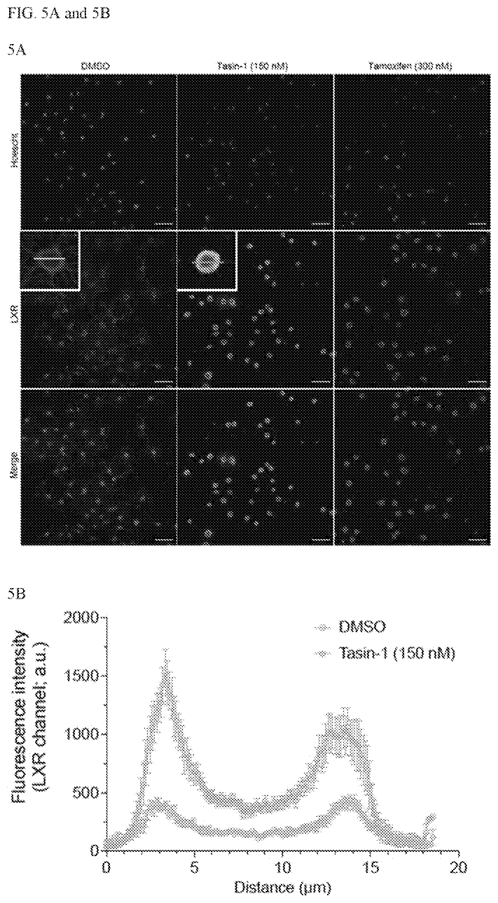

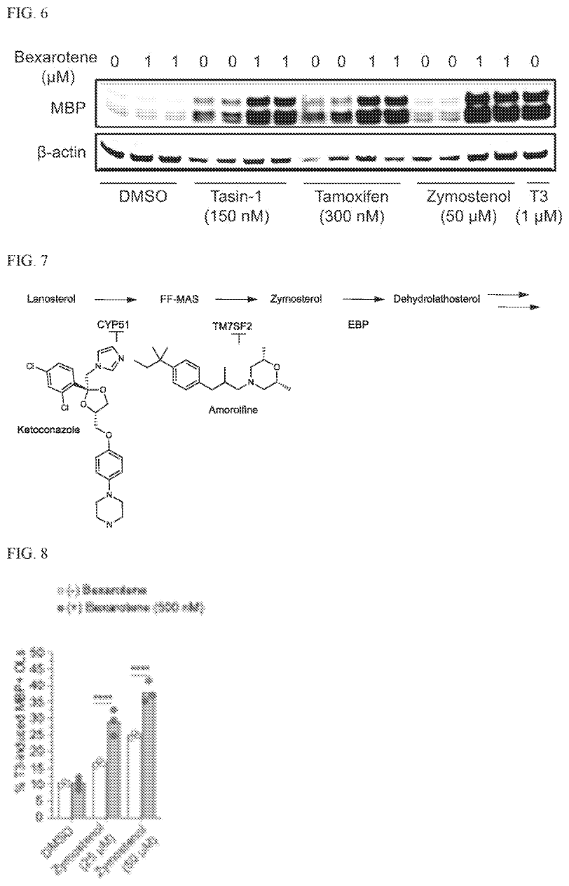

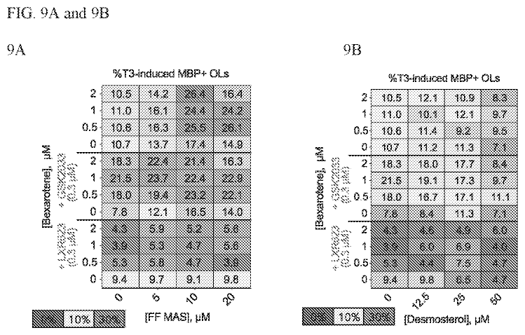

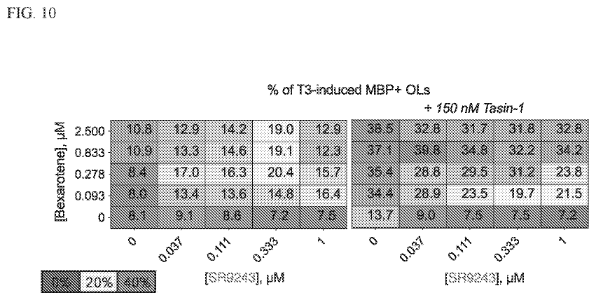

Upon activation, RXR can form heterodimers with other nuclear receptor(s) to stimulate a transcriptional profile that drives remyelination. We therefore aimed to identify the nuclear receptor binding partner of RXR that is targeted by endogenous metabolites resulting from CYP51, TM7SF2 and EBP inhibition to activate a pro-differentiation transcriptional program. We evaluated the impact of modulators of putative RXR binding partners on bexarotene+/−EBP inhibitors tasin-1 or tamoxifen. Addition of selective modulators of the liver X receptor (LXR) impacted the synergistic remyelinating activity of co-administration of bexarotene and agents that drive the accumulation of 8,9-unsaturated sterol intermediates (FIG. 4). Treatment with bexarotene and an LXR antagonist recapitulated, to a partial extent, the observed enhancement of % MBP+OLs following co-treatment with bexarotene and TASIN-1. The observed data demonstrates that tasin-1 or tamoxifen treatment with bexarotene results in the formation of products that regulate LXR, likely in an antagonistic manner. Our immunofluorescence analysis of LXR reveals a significant shift in localization of LXR following treatment with tasin-1 or tamoxifen, as evident by confocal-based imaging and a line profile analysis of LXR intensity bisecting individual oligodendrocytes following tasin-1 treatment (FIG. 5).

Synergism. Synergistic enhancement was illustrated by bexarotene-induced differentiation by tasin-1 or tamoxifen. When combined with the approved drug tamoxifen, the cell-based OPC differentiation ECₘₐₓ of bexarotene is ˜150 nM. The predicted Cₘₐₓ associated with the dose of bexarotene used in the CCMR One trial is 2.6 μM¹¹. The inventive combination thus results in an ˜10-fold increase in the overall level of observed OL maturation when compared to either agent alone.

Tamoxifen is a well-established nonsteroidal chemopreventive breast cancer drug. A 20 mg daily dose of tamoxifen is generally very well tolerated and is associated with a steady state plasma concentration of 834 nM (peak synergistic OPC activity observed at 200 nM) and accumulates in the CNS¹¹⁹. The concentration of bexarotene required to achieve optimal activity in the context of this combination (˜150 nM) is below that of the predicted exposure level of the CCMR One trial. Bexarotene demonstrates synergy exclusively with compounds that induce OL maturation via inhibition of cholesterol biosynthetic enzymes and specific sterol accumulation.

Useful references, including numbered references appearing throughout the present disclosure, are incorporated herein in their entireties, and they are as follows:
1 Chang, A., Tourtellotte, W. W., Rudick, R. & Trapp, B. D. Premyelinating oligodendrocytes in chronic lesions of multiple sclerosis. The New England journal of medicine 346, 165-173, doi:10.1056/NEJMoa010994 (2002).
2 Feinstein, A., Freeman, J. & Lo, A. C. Treatment of progressive multiple sclerosis: what works, what does not, and what is needed. The Lancet. Neurology 14, 194-207, doi:10.1016/s1474-4422(14)70231-5 (2015).
3 Franklin, R. J. & Gallo, V. The translational biology of remyelination: past, present, and future. Glia 62, 1905-1915, doi:10.1002/glia.22622 (2014).
4 Johnson, C. H., Ivanisevic, J. & Siuzdak, G. Metabolomics: beyond biomarkers and towards mechanisms. Nat Rev Mol Cell Biol 17, 451-459, doi:10.1038/nrm.2016.25 (2016).
5 Shyh-Chang, N. & Ng, H.-H. The metabolic programming of stem cells. Genes & development 31, 336-346, doi:10.1101/gad.293167.116 (2017).
6 Beyer, B. A. et al. Metabolomics-based discovery of a metabolite that enhances oligodendrocyte maturation. Nature chemical biology 14, 22-28, doi:10.1038/nchembio.2517 (2018).
7 Mei, F. et al. Micropillar arrays as a high-throughput screening platform for therapeutics in multiple sclerosis. Nature medicine 20, 954-960, doi:10.1038/nm.3618 (2014).
8 Deshmukh, V. A. et al. A regenerative approach to the treatment of multiple sclerosis. Nature 502, 327-332, doi:10.1038/nature12647 (2013).
9 Huang, J. K. et al. Retinoid X receptor gamma signaling accelerates CNS remyelination. Nat Neurosci 14, 45-53, doi:10.1038/nn.2702 (2011).
10 Green, A. J. et al. Clemastine fumarate as a remyelinating therapy for multiple sclerosis (ReBUILD): a randomised, controlled, double-blind, crossover trial. Lancet (London, England) 390, 2481-2489, doi:10.1016/s0140-6736(17)32346-2 (2017).
11 Brown, J. W. L. et al. Safety and efficacy of bexarotene in patients with relapsing-remitting multiple sclerosis (CCMR One): a randomised, double-blind, placebo-controlled, parallel-group, phase 2a study. The Lancet. Neurology 20, 709-720, doi:10.1016/S1474-4422(21)00179-4 (2021).
12 Pouly, S. & Antel, J. P. Multiple sclerosis and central nervous system demyelination. Journal of autoimmunity 13, 297-306, doi:10.1006/jaut.1999.0321 (1999).
13 Reich, D. S., Lucchinetti, C. F. & Calabresi, P. A. Multiple Sclerosis. The New England journal of medicine 378, 169-180, doi:10.1056/NEJMra1401483 (2018).
14 Loma, I. & Heyman, R. Multiple sclerosis: pathogenesis and treatment. Current neuropharmacology 9, 409-416, doi:10.2174/157015911796557911 (2011).
15 Rolak, L. A. Multiple sclerosis: it's not the disease you thought it was. Clin Med Res 1, 57-60 (2003).
16 Wallin, M. T. et al. The prevalence of MS in the United States: A population-based estimate using health claims data. Neurology 92, e1029-e1040, doi:10.1212/WNL.0000000000007035 (2019).
17 Paz-Zulueta, M., Paris-Bravo, P., Cantarero-Prieto, D., Blázquez-Fernández, C. & Oterino-Durán, A. A literature review of cost-of-illness studies on the economic burden of multiple sclerosis. Mult Scler Relat Disord 43, 102162, doi:10.1016/j.msard.2020.102162 (2020).
18 Gholamzad, M. et al. A comprehensive review on the treatment approaches of multiple sclerosis: currently and in the future. Inflamm Res 68, 25-38, doi:10.1007/s00011-018-1185-0 (2019).
19 Zawadzka, M. et al. CNS-resident glial progenitor/stem cells produce Schwann cells as well as oligodendrocytes during repair of CNS demyelination. Cell Stem Cell 6, 578-590, doi:10.1016/j.stem.2010.04.002 (2010).
20 Crawford, A. H., Tripathi, R. B., Richardson, W. D. & Franklin, R. J. M. Developmental Origin of Oligodendrocyte Lineage Cells Determines Response to Demyelination and Susceptibility to Age-Associated Functional Decline. Cell Rep 15, 761-773, doi:10.1016/j.celrep.2016.03.069 (2016).
21 Dawson, M. R., Polito, A., Levine, J. M. & Reynolds, R. NG2-expressing glial progenitor cells: an abundant and widespread population of cycling cells in the adult rat CNS. Mol Cell Neurosci 24, 476-488, doi:10.1016/s1044-7431(03)00210-0 (2003).
22 Franklin, R. J. & Ffrench-Constant, C. Remyelination in the CNS: from biology to therapy. Nature reviews. Neuroscience 9, 839-855, doi:10.1038/nrn2480 (2008).
23 Franklin, R. J. M. & Ffrench-Constant, C. Regenerating CNS myelin—from mechanisms to experimental medicines. Nature reviews. Neuroscience 18, 753-769, doi:10.1038/nrn.2017.136 (2017).
24 Kremer, D., Aktas, O., Hartung, H. P. & \ddot{K}ury, P. The complex world of oligodendroglial differentiation inhibitors. Ann Neurol 69, 602-618, doi:10.1002/ana.22415 (2011).
25 Patel, J. R. & Klein, R. S. Mediators of oligodendrocyte differentiation during remyelination. FEBS Lett 585, 3730-3737, doi:10.1016/j.febslet.2011.04.037 (2011).
26 Chari, D. M., Huang, W. L. & Blakemore, W. F. Dysfunctional oligodendrocyte progenitor cell (OPC) populations may inhibit repopulation of OPC depleted tissue. Journal of neuroscience research 73, 787-793, doi:10.1002/jnr.10700 (2003).
27 Wolswijk, G. Chronic stage multiple sclerosis lesions contain a relatively quiescent population of oligodendrocyte precursor cells. The Journal of neuroscience: the official journal of the Society for Neuroscience 18, 601-609 (1998).
28 Hart, I. K., Richardson, W. D., Bolsover, S. R. & Raff, M. C. PDGF and intracellular signaling in the timing of oligodendrocyte differentiation. The Journal of cell biology 109, 3411-3417 (1989).
29 Billon, N., Tokumoto, Y., Forrest, D. & Raff, M. Role of thyroid hormone receptors in timing oligodendrocyte differentiation. Dev Biol 235, 110-120, doi:10.1006/dbio.2001.0293 (2001).
30 Kuhlmann, T. et al. Differentiation block of oligodendroglial progenitor cells as a cause for remyelination failure in chronic multiple sclerosis. Brain: a journal of neurology 131, 1749-1758, doi:10.1093/brain/awn096 (2008).
31 Tokumoto, Y. M., Tang, D. G. & Raff, M. C. Two molecularly distinct intracellular pathways to oligodendrocyte differentiation: role of a p53 family protein. EMBO J 20, 5261-5268, doi:10.1093/emboj/20.18.5261 (2001).
32 Fernandez, M. et al. Thyroid hormone administration enhances remyelination in chronic demyelinating inflammatory disease. Proceedings of the National Academy of Sciences of the United States of America 101, 16363-16368, doi:10.1073/pnas.0407262101 (2004).
33 Neely, S. A. et al. New oligodendrocytes exhibit more abundant and accurate myelin regeneration than those that survive demyelination. Nat Neurosci, doi:10.1038/s41593-021-01009-x (2022).
34 Orthmann-Murphy, J. et al. Remyelination alters the pattern of myelin in the cerebral cortex. eLife 9, doi:10.7554/eLife.56621 (2020).
35 Bacmeister, C. M. et al. Motor learning promotes remyelination via new and surviving oligodendrocytes. Nat Neurosci 23, 819-831, doi:10.1038/s41593-020-0637-3 (2020).
36 Najm, F. J. et al. Drug-based modulation of endogenous stem cells promotes functional remyelination in vivo. Nature 522, 216-220, doi:10.1038/nature14335 (2015).
37 Jakel, S. et al. Altered human oligodendrocyte heterogeneity in multiple sclerosis. Nature 566, 543-547, doi:10.1038/s41586-019-0903-2 (2019).
38 Marisca, R. et al. Functionally distinct subgroups of oligodendrocyte precursor cells integrate neural activity and execute myelin formation. Nat Neurosci 23, 363-374, doi:10.1038/s41593-019-0581-2 (2020).
39 Neumann, B. et al. Metformin Restores CNS Remyelination Capacity by Rejuvenating Aged Stem Cells. Cell Stem Cell 25, 473-485.e478, doi:10.1016/j.stem.2019.08.015 (2019).
40 Hampton, D. W. et al. Focal immune-mediated white matter demyelination reveals an age-associated increase in axonal vulnerability and decreased remyelination efficiency. Am J Pathol 180, 1897-1905, doi:10.1016/j.ajpath.2012.01.018 (2012).
118 Lefebvre, P., Benomar, Y. & Staels, B. Retinoid X receptors: common heterodimerization partners with distinct functions. Trends Endocrinol Metab 21, 676-683, doi:10.1016/j.tem.2010.06.009 (2010).
119 Lien, E. A., Solheim, E. & Ueland, P. M. Distribution of tamoxifen and its metabolites in rat and human tissues during steady-state treatment. Cancer Res 51, 4837-4844 (1991).
120 Nelissen, K. et al. Liver X receptors regulate cholesterol homeostasis in oligodendrocytes. Journal of neuroscience research 90, 60-71, doi:10.1002/jnr.22743 (2012).
121 Yang, C. et al. Sterol intermediates from cholesterol biosynthetic pathway as liver X receptor ligands. J Biol Chem 281, 27816-27826, doi:10.1074/jbc.M603781200 (2006).
122 Meffre, D. et al. Liver X receptors alpha and beta promote myelination and remyelination in the cerebellum. Proceedings of the National Academy of Sciences of the United States of America 112, 7587-7592, doi:10.1073/pnas.1424951112 (2015).

We claim:

1. A method for the treatment of a demyelinating disease in a subject suffering therefrom, comprising administering to the subject at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof.

2. The method according to claim 1, wherein the RXRγ agonist is administered with an EBP inhibitor.

3. The method according to claim 1 or 2, wherein the EBP inhibitor is selected from tasin-1 and tamoxifen.

4. The method according to claim 1, wherein the RXRγ agonist is administered with a CYP51 inhibitor.

5. The method according to claim 4, wherein the CYP51 inhibitor is ketoconazole.

6. The method according to claim 1, wherein the RXRγ agonist is administered with a TM7SF2 inhibitor.

7. The method according to claim 6, wherein the TM7SF2 inhibitor is amorolfine.

8. The method according to any of claims 1-7, wherein the RXRγ agonist is administered with an LXR antagonist.

9. The method according to claim 8, wherein the LXR antagonist is GSK2033, LXR623, or SR9243.

10. The method according to any of claims 1-9, wherein the RXRγ agonist is bexarotene.

11. The method according to any of claims 1-10, wherein the demyelinating disease is a demyelinating disease of the central nervous system.

12. The method according to claim 11, wherein the demyelinating disease of the central nervous system is selected from multiple sclerosis, neuromyelitis optica (Devic's disease), an idiopathic inflammatory demyelinating disease, a leukodystrophic disease, acute disseminated encephalomyelitis, optic neuritis, transverse myelitis, adrenoleukodystrophy, adrenomyeloneuropathy, central pontine myelinolysis, and a leukoencephalopathy.

13. The method according to any of claims 1-12, wherein the demyelinating disease is multiple sclerosis.

14. The method according to any of claims 1-10, wherein the demyelinating disease is a demyelinating disease of the peripheral nervous system.

15. The method according to claim 14, wherein the demyelinating disease of the peripheral nervous system is selected from Guillain-Barre syndrome, chronic inflammatory demyelinating polyneuropathy, anti-MAG peripheral neuropathy, Charcot-Marie-Tooth disease, Hereditary neuropathy with liability to pressure palsy, a copper deficiency-associated condition, and progressive inflammatory neuropathy.

16. The method according to claim 15, wherein the copper deficiency-associated condition is selected from peripheral neuropathy, myelopathy, and optic neuropathy.

17. The method according to any of claims 1-16, wherein the treatment comprises retarding the rate of disease progression, arresting disease progression, reversing disease progression, or reducing the frequency and/or severity of disease symptoms.

18. A method for remyelination of demyelinated axons in a subject, comprising administering to the subject at least one Retinoid X receptor gamma (RXRγ) agonist and at least one member selected from a Liver X Receptor (LXR) antagonist, CYP51 inhibitor, TM7SF2 inhibitor, EBP inhibitor, and combinations thereof.

19. The method according to claim 18, wherein the axons are partially demyelinated.

20. The method according to claim 18, wherein the axons are completely demyelinated.

21. The method according to any of claims 18-20, wherein the axons are in the central nervous system of the subject.
